(19) United States
(12) Patent Application Publication
(10) Pub. No.: US 2011/0201908 A1
(43) Pub. Date: Aug. 18, 2011

(54) INTERMITTENT EXTRACORPOREAL SPECTROPHOTOMETRY

(76) Inventor: Joshua D. Farkas, Albany, NY (US)

(21) Appl. No.: 12/723,072

(22) Filed: Mar. 12, 2010

Related U.S. Application Data
(60) Provisional application No. 61/305,322, filed on Feb. 17, 2010.

(51) Int. Cl. A61B 5/1455

(52) U.S. Cl. 600/322

(57) ABSTRACT

An apparatus for intermittent measurement of blood parameters using spectrophotometry is provided. In exemplary embodiments, blood is temporarily withdrawn from the patient and passed through a cuvette, allowing spectrophotometric analysis. This blood may then immediately returned to the patient in a sterile fashion. The technique allows for real-time analysis of blood at the bedside without delays in transportation and laboratory analysis. In exemplary embodiments, there is no blood loss, so measurements can be repeated frequently with no detriment to the patient. In exemplary embodiments, the spectrophotometer is detachable from the cuvette and does not come in contact with blood, such that it can be used for multiple patients with minimal cost. The apparatus may be used to measure the oxygen saturation of blood and hemoglobin concentration, although it could be easily adapted to measure these and many other parameters simultaneously.

### CROSS REFERENCE TO RELATED APPLICATIONS

The present application claims priority to U.S. Provisional Patent Application Ser. No. 61/305,322, filed Feb. 17, 2010, the entire contents of which are specifically incorporated by reference herein.

### FIELD

The present disclosure relates to an apparatus and method for intermittently removing blood from a patient for spetrophotometric analysis in a rapid and safe manner. Multiple blood parameters can be determined quickly and simultaneously, including oxygen saturation and hemoglobin concentration.

### BACKGROUND

With advances in optical techniques and data analysis, the potential utility of spectrophotometric analysis of blood is expanding. Technology has long existed for measurement of parameters including hemoglobin concentration, oxygen saturation, carboxyhemoglobin concentration, and methemoglobin concentration. More recently techniques have been developed to measure additional blood parameters including blood urea nitrogen and blood osmolarity (U.S. Pat. No. 6,687,519 B2). With ongoing advances it is likely that an increasing number of parameters will become measurable in this fashion. A single spectrophotometer could measure all of these parameters almost instantaneously.

Spectrophotometric analysis of blood has enormous potential for application at the bedside in clinical medicine. It provides rapid results in real time, without requiring reagents or additional processing of blood. Noninvasive measurement of arterial blood oxygen saturation based on fluctuations in absorption (for example of light passed through a finger) allowed blood oxygen saturation to be easily measured, revolutionizing clinical assessment of blood oxygenation. There are a variety of devices available which use spectrophotometry to monitor the oxygen saturation and hemoglobin concentration of blood passing through extracorporeal circuits such as during hemodialysis or cardio-pulmonary bypass (i.e., U.S. Pat. No. 6,144,444 and U.S. Pat. No. 6,746,415 B1).

As important as arterial oxygen levels are, an argument could be made that mixed venous oxygen saturation is an even more important measurement. Mixed venous oxygen saturation refers to the oxygen level in the blood returning to the heart from systemic circulation (measured via an indwelling central venous catheter lying in the superior vena cava or a Swan-Ganz catheter lying in the pulmonary artery). Low levels of mixed venous oxygen saturation typically indicate shock states wherein the amount of blood circulated to the tissues is low, forcing tissues to extract as much oxygen from the blood as possible. The utility of mixed venous oxygen saturation in shock states has recently been recognized in guidelines for the management of septic shock, which involve repeatedly measuring the mixed venous oxygen saturation and aggressively resuscitating the patient until this value is normalized (Dellinger et al.).

Unfortunately, currently it is difficult to apply guidelines for the management of septic shock because it is difficult to monitor mixed venous oxygen saturation in real time. Special indwelling central venous catheters incorporating a fiberoptic channel for reflectance spectrophotometry have been developed to allow continuous monitoring of mixed venous oxygen saturation, but their high cost has prevented widespread use. Mixed venous blood may be removed from the patient via an indwelling catheter and sent to the laboratory, but subsequent delays in transport and laboratory processing are problematic if immediate management decisions are required.

Accordingly, there is a need in the art to monitor mixed venous oxygen saturation and other blood parameters in more efficient fasion.

### SUMMARY OF INVENTION

The present invention overcomes and alleviates the above described and other problems in the art by providing an apparatus and method for temporary removal of blood from a patient into a cuvette, allowing for spectrophotometric analysis at the bedside in real time. In exemplary embodiments, this invention allows for rapid analysis of mixed venous blood in the resuscitation of patients with shock. Also, there are a wide range of potential applications of this invention to perform serial, inexpensive, real-time spectrophotometric analysis of venous or arterial blood with no blood loss required.

In an exemplary embodiment, the present apparatus comprises two main components: a cuvette attached to an indwelling catheter and a detachable spectrophotometer. In an exemplary embodiment, the cuvette component is attached to an indwelling catheter. The cuvette may remain attached to the catheter indefinitely, and drugs or fluids may be infused through the cuvette when it is not being used to analyze the blood. To analyze blood, a vacuum source, such as a syringe or pump, may be used to draw blood from the body into the cuvette temporarily. This blood could be analyzed rapidly and subsequently reinfused into the patient to avoid any blood loss. In such an exemplary embodiment, the entire procedure of blood withdrawal, analysis, and reinfusion could be done rapidly at the bedside by a nurse or similar healthcare professional.

In an exemplary embodiment, the spectrophotometer component includes a spectrophotometer designed such that the cuvette could be inserted into it. This could be connected to (or include) an analysis unit with the computing power necessary to analyze the absorption spectrum and display results immediately. In such an embodiment, the spectrophotometer would never come in contact with blood, and would only be required briefly and intermittently for use with a single patient. As such, a single spectrophotometer would be adequate to perform intermittent extracorporeal spectrophotometry on a group of patients (for example, 10-20 patients in an intensive care unit).

### BRIEF DESCRIPTION OF THE DRAWINGS

Referring now to the drawings, wherein like elements are numbered alike in the following FIGURES:

FIG. 1 is a schematic showing an exemplary cuvette and spectrophotometer system coupled to an indwelling vascular catheter;

FIG. 2 is a perspective view of the exemplary apparatus in FIG. 1;

FIG. 3 is a schematic illustrating an exemplary intravenous infusion line connected to an exemplary cuvette and central venous catheter;

FIG. 4 is a schematic illustrating an exemplary syringe connected to an exemplary cuvette and central venous catheter;

FIG. 5 is a schematic illustrating an exemplary withdrawal of blood and/or infused fluid via an exemplary syringe connected to an exemplary cuvette and central venous catheter;

FIG. 6 is a schematic illustrating an exemplary spectrophotometer analaysis of blood provided within an exemplary cuvette connected to a central venous catheter;

FIG. 7 is a schematic illustrating exemplary re-infusion of blood and/or infused fluid via an exemplary syringe connected to an exemplary cuvette and central venous catheter; and

FIG. 8 shows an exemplary embodiment of the present apparatus, wherein an exemplary cuvette is inserted within an indwelling catheter itself (the type of indwelling catheter shown here is a triple-lumen central venous catheter, but application would not be limited to this device).

### DETAILED DESCRIPTION OF THE INVENTION

As is noted above, the present invention provides an apparatus and method for temporary removal of blood from a patient into a cuvette, allowing for spectrophotometric analysis at the bedside in real time. In exemplary embodiments, this invention allows for rapid analysis of mixed venous blood in the resuscitation of patients with shock. Also, there are a wide range of potential applications of this invention to perform serial, inexpensive, real-time spectrophotometric analysis of venous or arterial blood with no blood loss required.

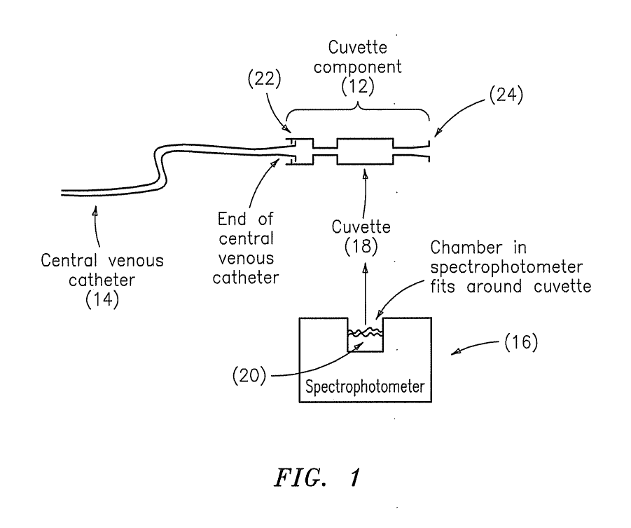

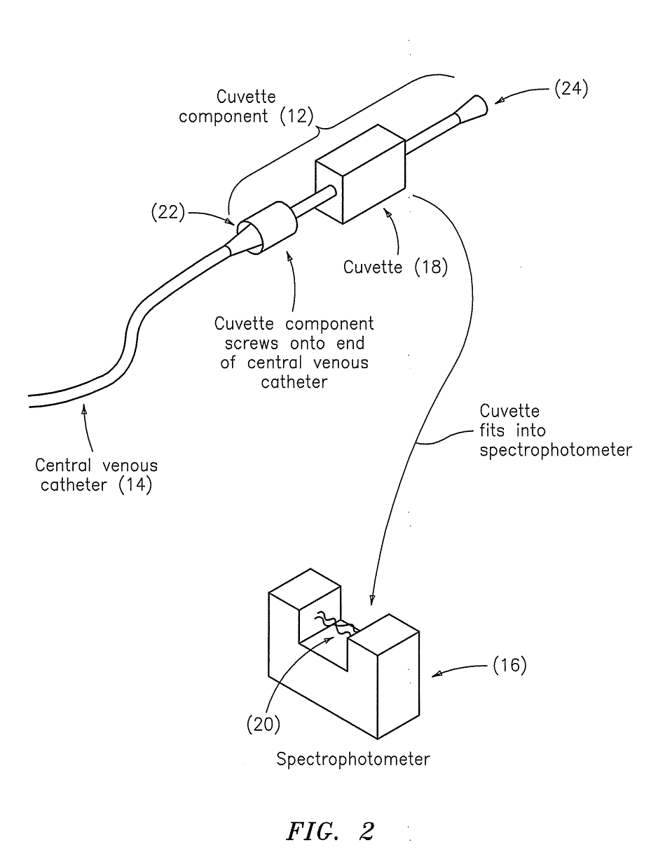

Referring now to FIGS. 1 and 2, in an exemplary embodiment, the present apparatus comprises two main components: a cuvette component 12 attached to an indwelling catheter 14 and a detachable spectrophotometer 16. The cuvette component 12 includes a hollow blood sampling portion 18 (which may, e.g., be a transparent windowed cavity having a shape that is complementary to a dimension of the spectrophotometer sampling area 20) and first 22 and second 24 attachment portions configured to engage the indwelling catheter 14 at the first attachment portion 22 and an external device (e.g., infusion line, syringe, etc) at the second attachment portion 24. In an exemplary embodiment, the cuvette component is attached to an indwelling catheter (FIGS. 1-2). The cuvette may remain attached to the catheter indefinitely, and drugs or fluids may be infused (e.g., via exemplary intravenous infusion line 26 in FIG. 3) through the cuvette when it is not being used to analyze the blood.

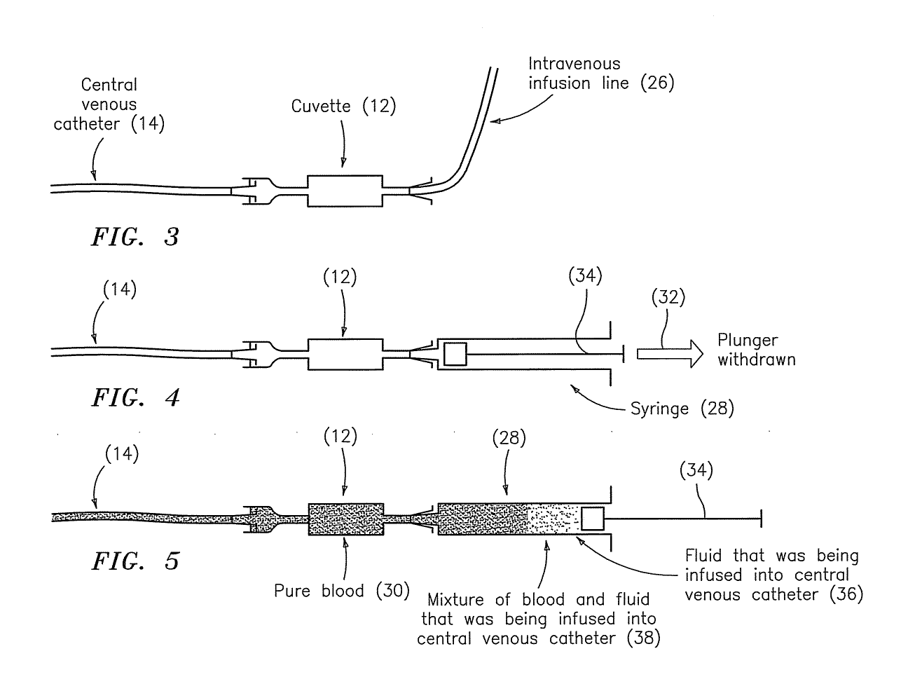

Referring now to FIGS. 4-6, to analyze blood, a syringe 28 may be used to draw blood 30 from the body into the cuvette 12 temporarily. This blood could be analyzed rapidly and subsequently reinfused into the patient to avoid any blood loss. In such an exemplary embodiment, the entire procedure of blood withdrawal, analysis, and reinfusion could be done rapidly at the bedside by a nurse or similar healthcare professional. FIG. 4 illustrates the beginning of withdrawal (see arrow 32) of a plunger 34 of a syringe 28. FIG. 5 illustrates an exemplary configuration where pure blood 30 is present in the cuvette 12 and both infusion fluid 36 and a mixture 38 of blood and infusion fluid are present within the syringe 28. FIG. 6 illustrates exemplary spectrophotometer analysis of the blood 30 within the cuvette 12.

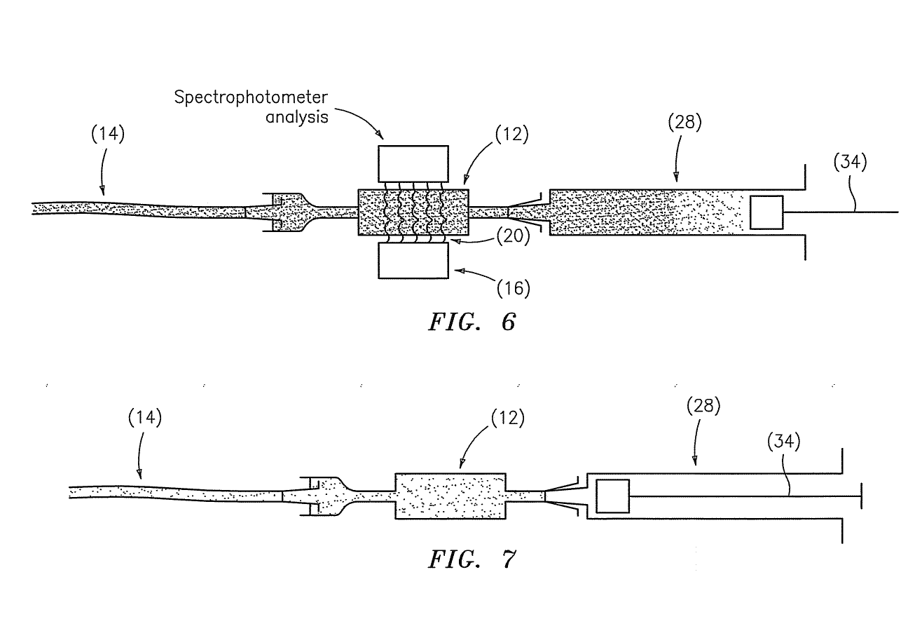

Referring to FIG. 7, subsequent exemplary replacement of the syringe plunger 34 to a contracted position results in re-infusion of blood and infusion fluid into the catheter.

In an exemplary embodiment, the spectrophotometer component includes a spectrophotometer designed such that the cuvette could be inserted into it. FIGS. 1-2 and 6 illustrate an exemplary cuvette 12 and spectrophotometer 20 arrangement. The spectrophotometer could be connected to (or include) an analysis unit with the computing power necessary to analyze the absorption spectrum and display results immediately. In such an embodiment, the spectrophotometer would never come in contact with blood, and would only be required briefly and intermittently for use with a single patient. As such, a single spectrophotometer would be adequate to perform intermittent extracorporeal spectrophotometry on a group of patients (for example, 10-20 patients in an intensive care unit).

### Cuvette Component

An exemplary cuvette component comprises (without limitation) hard plastic. In exemplary embodiments, on the first end portion 24, it could screw onto (or otherwise attach to) the end of the indwelling catheter (see, e.g, FIGS. 1-2). The center could consist of a rectangular (or other shaped) transparent portion which would function as a cuvette (as used herein, the term “cuvette” is not intended to specify any particular material or configuration, the important aspect being that the cuvette provides a blood sampling volume that isolates the blood sample from the spectrophotometer and is compatible with a sampling area of a spectrophotometer). Exemplary embodiments include a central portion having clear walls which would allow spectrophotometric analysis of the fluid within the plastic. On a right end portion 24, the device may, e.g., be shaped identically to the end of the indwelling catheter to allow for standard connections of peripheral devices just as an indwelling catheter would. Thus, the right end 24, then, could be attached to, e.g., a stopper or intravenous line in the same manner that an indwelling catheter could be attached.

In exemplary embodiments, the cuvette would not have to be changed every time blood parameters were measured. Instead, the cuvette could remain attached to the indwelling catheter indefinitely. When the cuvette was not being used to sample blood, the cuvette and indwelling catheter assembly could be used in a manner identical to an unmodified indwelling catheter. For example, drugs or fluids could be infused through the cuvette and indwelling catheter (FIG. 3). Therefore, in between measurements the venous catheter would be fully functional, rather than having one port permanently dedicated to blood analysis. The cuvette material Could be noncorrosive and inert such that infusion of drugs or intravenous fluids would be safe.

In exemplary embodiments, the cuvette would be attached to a central venous catheter and could be used to measure the mixed venous oxygen saturation (the oxygen saturation of blood in the superior vena cava). However, the cuvette could also be connected to any indwelling catheter (for example, a peripherally-inserted central catheter aka PICC line, arterial catheter, or Swan-Ganz catheter). If the invention were being used primarily to monitor resuscitation of a patient in shock then it would be important for the cuvette to be attached to a catheter lying in the central venous system or pulmonary artery (i.e., a central venous catheter in the internal jugular vein or subclavian vein, or a Swan-Ganz catheter) to allow for measuring of the mixed venous oxygen saturation. However, if the invention was being used to monitor a blood parameter such as hemoglobin concentration (which is constant throughout the body) then it could be attached to any indwelling catheter within any artery or vein.

As noted earlier, arterial blood oxygen saturation can typically be measured noninvasively with a spectrophotometer attached to the finger. However, in some patients who are in shock or have poor peripheral circulation, this approach fails to provide reliable data. In this situation, the invention could be connected to an indwelling arterial catheter to provide reliable measurement of the arterial oxygen saturation.

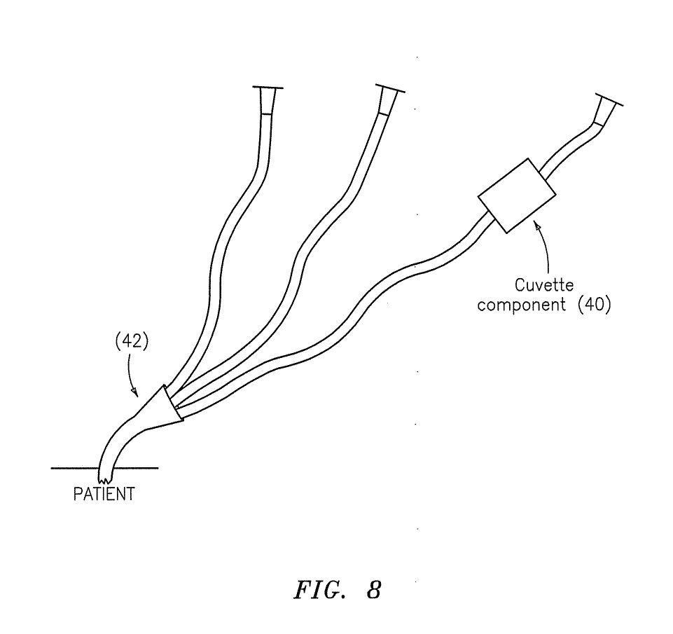

In other exemplary embodiments, the cuvette component could be built into the indwelling vascular catheter. For example, FIG. 8 shows an exemplary cuvette component 40 permanently built into an exemplary triple-lumen central venous catheter 42. It should be noted that a cuvette could also be built into any indwelling venous catheter (i.e., Swan-Ganz catheter, other type of central venous catheter, or arterial catheter). Also, if a cuvette size was standardized, then the same spectrophotometer could be used with both cuvettes attached to indwelling catheters, as well as cuvettes permanently built into indwelling catheters.

### Spectrophotometer Component

The second component would be a spectrophotometer. In exemplary embodiments, the spectrophotometer could be portable. In other exemplary embodiments, the spectrophotometer could be specially designed to fit around the cuvette and measure the absorption of fluid within the cuvette. The spectrophotometer could be programmed to measure the absorption spectra of blood within the cuvette and calculate relevant parameters from this spectrum (i.e. oxygen saturation, hemoglobin concentration).

The spectrophotometer would not come in direct contact with blood, so the spectrophotometer could be reused indefinitely without requiring any extensive sterilization or extensive cleaning (the surface of the spectrophotometer may be rapidly wiped with alcohol to prevent cross-contamination in between different patients, similar to other durable medical equipment). Since spectrophotometric analysis requires only a few seconds and is only required intermittently, a single spectrophotometer could be used to service an entire intensive care unit. By allowing the spectrophotometer to be detached from the cuvette and thus allowing one spectrophotometer to service an entire intensive care unit on an indefinite basis, the cost of this technology would be reduced substantially.

### Method for Performing Intermittent Extracorporeal Spectrophotometry

In exemplary embodiments, to perform extracorporeal spectrophotometry, first a syringe 28 could be attached to the cuvette 12 and blood 30 could be drawn back into the syringe 28 (See FIGS. 4-5). In exemplary embodiments, the initial fluid withdrawn would be fluid being infused into the indwelling catheter but eventually this would result in withdrawing blood. The final result would be a mixture 38 of diluted blood within the syringe 28, and a pure solution of blood 30 within the cuvette 12 (See FIG. 5).

The cuvette 12 could then be slipped into the portable spectrophotometer 16 to allow for spectrophotometeric analysis of the blood 30 (see FIG. 6). Such analysis could be performed rapidly at the bedside with immediate results displayed on the spectrophotometer. The spectrophotometer could be slipped off the cuvette when analysis was completed. In exemplary embodiments the contents of the syringe could also be injected back into the cuvette (See FIG. 7). This would prevent any loss of blood associated with the procedure. The cuvette could then be flushed to remove any residual blood from the cuvette or indwelling catheter.

Recent consensus guidelines have emphasized that it is important to minimize the amount of blood which is removed from patients due to repeated laboratory studies. Intermittent extracorporeal spectrophotometry would have the ability to perform frequent measurement of important blood parameters without requiring frequent blood loss.

### Additional Embodiments

Exemplary embodiments described above comprise attaching the cuvette 12 to a central venous catheter 14 to allow for measurement of mixed venous oxygen saturation to

facilitate resuscitation of a patient in shock. However, the utility of this invention is not limited to this application. One alternative embodiment of the invention would be to monitor hemoglobin concentration over time in a hemorrhaging patient. Another alternative embodiment of the invention would be to determine arterial oxygen saturation in a patient who has poor perfusion such that noninvasive oxygen measurement is unreliable (as discussed above). With advances in various types of spectroscopy and computer data analysis the applications of this invention will likely expand over time. It should be noted that the spectrophotometer could measure multiple parameters simultaneously (i.e., a single device could measure mixed venous oxygen saturation, hemoglobin level, and a variety of blood chemistries).

Also, in certain exemplary embodiments, a syringe is used to withdraw blood from the patient. However, other devices could be used to temporarily withdraw blood from the patient to fill a cuvette, for example by alternate mechanisms of applying a vacuum. In one such exemplary embodiment, an infusion pump (for example, one that was being used for continuous infusion of intravenous fluids into the patient) could be used to withdraw blood from the patient. In exemplary embodiments, the infusion pump could be programmed (or otherwise manipulated) to temporarily reverse direction and withdraw a small amount of blood from the patient (sufficient to fill the cuvette with blood) and then the pump would pause. After spectrophotometry was performed, the pump would then again reverse direction in order to resume infusion of fluid into the patient (and, in so doing, flush residual blood from the catheter and cuvette). In such an exemplary embodiment, manipulation of the catheter could be minimized, which would minimize the risk of introducing infection. A secondary advantage of this exemplary embodiment is that it would not require disposable materials (reducing cost) and may be easier for nursing or other ancillary staff to perform.

It will be apparent to those skilled in the art that, while exemplary embodiments have been shown and described, various modifications and variations can be made to the apparatus and method for temporary removal of blood from a patient into a cuvette for spectrophotometric analysis disclosed herein without departing from the spirit or scope of the invention. Accordingly, it is to be understood that the various embodiments have been described by way of illustration and not limitation.

What is claimed is:

1. An apparatus for intermittent extracorporeal spectrophotometry of blood in a patient comprising:
an indwelling catheter configured to operatively communicate with the vasculature of a patient;
a spectrophotometer analysis component; and
a cuvette component operatively associated with said spectrophotometer analysis component and said indwelling catheter, wherein said cuvette component is configured to receive a blood sample withdrawn from said indwelling catheter, and wherein said cuvette component and a sampling portion of said spectrophotometer analysis component are configured such that the cuvette component may be inserted into said spectrophotometer analysis component and such that the spectrophotometer analysis component may test said blood without exposure to said blood.

2. An apparatus in accordance with claim 1, wherein said spectrophotometer analysis component is a portable spectrophotometer.

3. An apparatus in accordance with claim 1, wherein said spectrophotometer analysis component and said cuvette are configured with complementary dimensions to allow rapid insertion of the cuvette into and removal of the cuvette from the sampling portion of said spectrophotometer analysis component.

4. An apparatus in accordance with claim 1, further comprising a syringe configured to operatively communicate with said cuvette, wherein the syringe is further configured to withdraw blood from said catheter and into said cuvette for sampling by said spectrophotometer analysis component.

5. An apparatus in accordance with claim 4, wherein said syringe is configured to re-infuse said blood through said catheter.

6. A method for intermittent extracorporeal spectrophotometry of a blood sample in a cuvette, comprising:
providing an indwelling catheter in communication with the vasculature of a patient;
providing a cuvette component in communication with said catheter;
withdrawing a blood sample through said indwelling catheter and into said cuvette;
providing a spectrophotometer analysis component configured to receive and release said cuvette and to sample blood provided within said cuvette;
sampling blood provided within said cuvette; and
releasing said cuvette from said spectrophotometer analysis component.

7. The method of claim 6, further comprising reinfusing said blood within said cuvette through said indwelling catheter and into the patient.

8. The method of claim 6, wherein withdrawing a blood sample through said indwelling catheter and into said cuvette comprises applying a vacuum generated by withdrawing the plunger of a syringe in operative communication with said cuvette.
